(19) United States
(12) Patent Application Publication
(10) Pub. No.: US 2022/0152156 A1
(43) Pub. Date: May 19, 2022

(54) METHOD OF USING PEGYLATED INTERFERON-ALPHA

(71) Applicant: PharmaEssentia Corporation, Taipei (TW)

(72) Inventors: Ko-Chung Lin, Lexington, MA (US); Jean-Jacques Kiladjian, Paris (FR)

(21) Appl. No.: 17/525,471

(22) Filed: Nov. 12, 2021

Related U.S. Application Data
(60) Provisional application No. 63/113,534, filed on Nov. 13, 2020.

(51) Int. Cl. A61K 38/21; A61K 47/60; A61P 7/00

(52) U.S. Cl. CPC A61K 38/212; A61P 7/00; A61K 47/60

(57) ABSTRACT

A method of treating a myeloid neoplasm in a subject, the method comprising administering to a subject in need thereof a 50 to 500 μg dose of a pegylated interferon-α once every 2 to 8 weeks, the pegylated interferon-α being a conjugate of formula I:

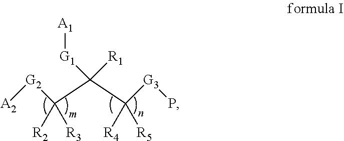

in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10;
wherein the subject has a complete molecular response, the complete molecular response including a JAK2617F allele burden below 1%.

### CROSS-REFERENCE

This application claims priority to U.S. Provisional Application No. 63/113,534, filed on Nov. 13, 2020, the entire content of which is hereby incorporated by reference herein.

### BACKGROUND

Myeloproliferative neoplasms include polycythemia vera, essential thrombocythemia and myelofibrosis. These diseases are characterized by uncontrolled malignant proliferation of hematopoietic cells leading to increased concentrations of red cells, often accompanied by elevated leucocyte and platelet counts. The oncogenic pathway of these diseases is driven by the Val617Phe mutation in the JAK2 gene, which results in constitutive kinase activity promoting both hematopoietic cell proliferation and a proinflammatory state. Patients are predisposed to thrombosis and disease progression to leukemia, which result in poor prognosis.

Hydroxyurea (HU), an unspecific antiproliferative ribonucleotide reductase inhibitor, is a standard therapy for patients needing cytoreductive treatment. However, a significant number of patients are either intolerant of HU because of hematologic or nonhematologic toxicity or resistant because of a lack of effective cytoreduction treatment with HU.

Interferons belong to the large class of glycoproteins known as cytokines. Interferons are named for their ability to “interfere” with viral replication by protecting cells from virus infection. More than twenty distinct interferon genes and proteins have been identified in animals, including humans. They are typically divided among three classes: Type I interferon, Type II interferon, and Type III interferon. Interferons of all three classes are important for fighting viral infections and regulating the immune system.

### SUMMARY

In one aspect, described herein is a method of treating a myeloid neoplasm in a subject, the method comprising administering to a subject in need thereof a 50 to 500 μg dose of a pegylated interferon-α once every 2 to 8 weeks, the pegylated interferon-α being a conjugate of formula I:

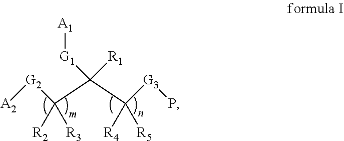

in which

each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;

each of A₁ and A₂, independently, is a polymer moiety;

each of G₁, G₂, and G₃, independently, is a bond or a linking functional group;

P is an interferon-α moiety;

m is 0 or an integer of 1-10; and

n is an integer of 1-10;

wherein the subject has a complete molecular response, the complete molecular response including a JAK2617F allele burden below 1%.

In some embodiments, the JAK2617F allele burden is below 0.01%.

In some embodiments, the subject has the complete molecular response by 24 to 60 months (e.g., 24, 30, 36, 42, 48, 52 or 60 months) of being treated with the pegylated interferon-α. In some embodiments, the subject has the complete molecular response by 24 to 48 months of being treated with the pegylated interferon-α.

In some embodiments, the subject also has a complete hematological response by 24 to 60 months (e.g., 24, 30, 36, 42, 48, 52 or 60 months) of being treated with the pegylated interferon-α. In some embodiments, the subject has the complete molecular response and the complete hematological response simultaneously. The complete hematological response can include hematocrit <45% without phlebotomy for at least 3 months, platelet count <400×10⁹/L, and white blood cell count <10×10⁹ cells/L.

In some embodiments, the subject has a JAK2617F allele burden of at least 10% to 60% prior to the administration of the pegylated interferon-α.

In some embodiments, the subject is continuously treated with the pegylated interferon-α after exhibiting the complete molecular response. After the complete molecular response is achieved or detected, it can be maintained for at least one year or further improved (i.e., further reduction of the JAK2617F allele burden).

In some embodiments, the subject is treated with the pegylated interferon-α for at least 4 years. The subject can be treated with the pegylated interferon-α for at least 5 to 10 years.

In some embodiments, the subject is administered the pegylated interferon-α once every 2 to 4 weeks. In some embodiments, the subject is administered a 250 to 500 μg dose of the pegylated interferon-α once every 2 to 4 weeks.

In some embodiments, the subject is administered a starting dose of 50 to 350 μg once every 2 to 8 weeks, wherein the starting dose is increased incrementally until a target dose of 500 μg is reached. The target dose can be reached by 4 to 48 weeks (e.g., 4 to 8 weeks) from administration of the starting dose.

In some embodiments, the subject is initially administered the pegylated interferon-α once every 2 weeks for a first treatment period and switched to administration of the pegylated interferon-α once every 3 to 8 weeks for a second treatment period. In some embodiments, the subject exhibits the complete molecular response before or after the switch.

In some embodiments, the myeloid neoplasm is polycythemia vera, essential thrombocythemia or myelofibrosis.

In some embodiments, the conjugate of formula I has one or more properties including:

(i) a median Tmax in the range of 3 to 6 days following administration of multiple 50 to 540 μg doses of the conjugate once every two weeks to subjects;

(ii) a mean T_{1/2} in the range of 6 to 10 days following administration of multiple 50 to 540 μg doses of the conjugate once every two weeks to subjects; and

(iii) an individual maximum tolerated dose of at least 500 μg once every 2 to 4 weeks in subjects.

In some embodiments, the conjugate has one or more features including: G3 is a bond and P is an interferon-α moiety in which the amino group at the N-terminus is attached to G3; A₁ and A₂ are polyalkylene oxide moieties each having a molecular weight of 10-30 kD; each of G₁ and G₂ is

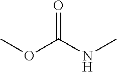

in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues. In some embodiments, the interferon-α moiety is a human interferon-α-2b having an extra proline residue at the N-terminus and is 166 amino acids in length.

In some embodiments, the conjugate is

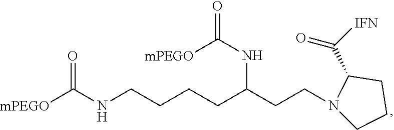

in which mPEG has a molecular weight of 20 kD and IFN is an interferon-α-2b.

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a set of graphs showing doses of ropeginterferon alpha-2b (μg) administered per 4-week period. Top graph: cumulative dose per 4-week period; bottom graph: administration schedule. Diamonds: mean; boxes: median (Q1-Q3).

FIG. 2 is a graph showing the percentages of phlebotomy-free patients over the study period.

FIG. 3 is a set of graphs showing rate of patients with JAK2V617F allele burden ≤50% and >50% per year during the study period.

FIG. 4 is a graph showing combined analysis of hematological and molecular parameters.

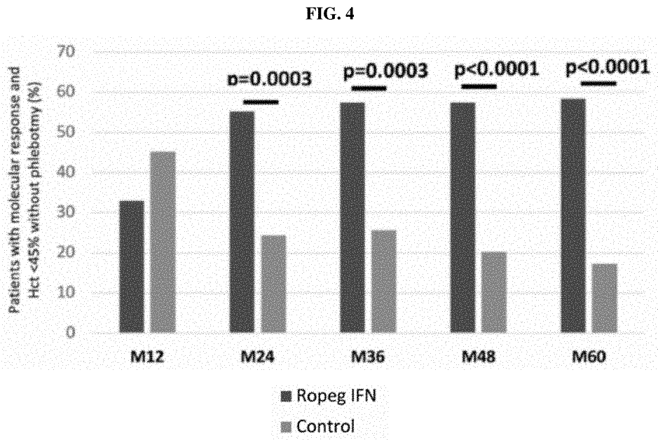

### DETAILED DESCRIPTION

Data described below demonstrated for the first time in a randomized study that, surprisingly, a pegylated interferon-α, e.g., ropeginterferon alpha-2b (also known as P1101 and AOP2014), can induce deep and durable molecular responses including complete molecular response in patients, which underscores its disease modifying potential. These results also suggest that patients can achieve operational cure (with both complete hematologic response and complete molecular response) with a pegylated interferon-α.

Accordingly, described herein are methods including administering a pegylated interferon-α to a patient to treat myeloproliferative neoplasms such as myelofibrosis, polycythemia vera and essential thrombocythemia.

A pegylated interferon-α used in any of the methods described herein can be a conjugate of formula I:

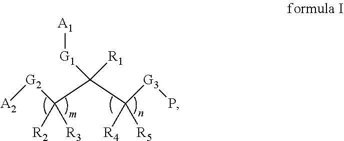

wherein each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl; each of A₁ and A₂, independently, is a polymer moiety; each of G₁, G₂, and G₃, independently, is a bond or a linking functional group; P is an interferon-α moiety; m is 0 or an integer of 1-10; and n is an integer of 1-10.

Referring to the above formula, the conjugate can have one or more of the following features: G3 is a bond and P is an interferon-α moiety (e.g., a human interferon-α-2b) in which the amino group at the N-terminus is attached to G3; A₁ and A₂ are polyalkylene oxide moieties having a molecular weight of 2-100 kD (preferably 10-30 kD), each of G₁ and G₂ is

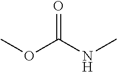

(in which O is attached to A₁ or A₂, and NH is attached to a carbon atom as shown in formula I), or each of G₁ and G₂ is urea, sulfonamide, or amide, (in which N is attached to a carbon atom as shown in formula I); m is 4, n is 2, and each of R₁, R₂, R₃, R₄, and R₅ is H; and the interferon-α moiety is a modified interferon-α moiety containing 1-4 additional amino acid residues. In some embodiments, the interferon-α moiety is a human interferon α-2b having an extra proline residue at the N-terminus and is 166 amino acids in length.

The conjugate can also have one or more of the following properties: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate is ropeginterferon alpha-2b (AOP2014/P1101), which has a predominant isoform having the formula:

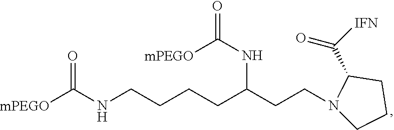

in which mPEG has a molecular weight of 20 kD and IFN is an interferon-α-2b (e.g., a human interferon-α-2b).

Ropeginterferon alpha-2b is produced by covalent attachment of a 40 kDa PEG molecule to the N-terminal proline residue of a Proline-Interferon-2b (Pro-IFN alpha-2b). Proline-interferon alpha-2b is generated by recombinant DNA technology introducing an extra proline residue to a human interferon α-2b at N-terminus, giving a polypeptide of total 166 amino acids in length. Pro-IFN alpha-2b has a molecular weight of approximately 19 kDa and has the amino acid sequence identical to the theoretical sequence predicted excluding the additional N-terminal proline. It is then PEGylated with an approximately 40 kDa PEG moiety forming approximately 60 kDa PEGylated proline-interferon alpha-2b or ropeginterferon alpha-2b. The biological activity of ropeginterferon alpha-2b is determined by cytopathic effect (CPE)-based antiviral assay.

The conjugate of formula I is described in detail in WO2009/023826A₁. In particular, WO2009/023826A₁ teaches a method of making AOP2014/P1101.

In any of the methods described herein, the pegylated interferon-α can be administered by any means known in the art, e.g., via subcutaneous or intravenous route. The pegylated interferon-α can be formulated as an injectable formulation. For example, it can be in the form of a ready-to-use prefilled syringe (PFS) containing, e.g., 0.2 to 2 mL of solution, that can be for self-injection. Each PFS can contain the labeled amount of the drug product, sodium chloride, sodium acetate anhydrous, acetic acid, benzyl alcohol, and polysorbate 80. The vehicle for the drug product can be sterile water for injection and the drug product solution can have a pH of about 6.0.

The term “dose” refers to the amount of a compound administered to a subject at one time.

The term “interval” refers to the time between administration of two consecutive doses. In any of the methods described herein, the pegylated interferon-α is administered at an interval of 2 to 8 weeks, e.g., 2, 3, 4, 5, 6, 7, or 8 weeks. For example, a dose can be administered once every 2, 3, 4, 5, 6, 7, or 8 weeks. An interval that is defined in days or months is also contemplated. A regular interval of 10 to 60 days (e.g., 14, 21, 25, 26, 27, 28, 29, 30, 31, 35, 42, 49, and 56 days), one month, or two months can be utilized in any of the methods.

A treatment period can be at least 1, 2, 3, 4, 5, 6, 7, 8, 9, 10, 11, 12, 24, 36, 42, 48, 54, 60, 66, 72, 78, 84 or more months. In some embodiments, the treatment period is 1, 2, 3, 4, 4.5, 5, 5.5, 6, 6.5, 7, 7.5, 8, 8.5, 9, 9.5, 10 or more years.

A dose administered during a treatment period ranges from 50 to 650 μg. The dose can be 50 μg, up to 55 μg, specifically up to 60 μg, specifically up to 65 μg, specifically up to 75 μg, specifically up to 80 μg, specifically up to 85 μg, specifically up to 90 μg, specifically up to 95 μg, specifically up to 100 μg, specifically up to 105 μg, specifically up to 110 μg, specifically up to 115 μg, specifically up to 120 μg, specifically up to 125 μg, specifically up to 130 μg, specifically up to 135 μg, specifically up to 140 μg, specifically up to 145 μg, specifically up to 150 μg, specifically up to 155 μg, specifically up to 160 μg, specifically up to 165 μg, specifically up to 170 μg, specifically up to 175 μg, specifically up to 180 μg, specifically up to 185 μg, specifically up to 190 μg, specifically up to 195 μg, specifically up to 200 μg, specifically up to 205 μg, specifically up to 210 μg, specifically up to 215 μg, specifically up to 225 μg, specifically up to 230 μg, specifically up to 235 μg, specifically up to 240 μg, specifically up to 245 μg, specifically up to 250 μg, specifically up to 255 μg, specifically up to 260 μg, specifically up to 265 μg, specifically up to 270 μg, specifically up to 275 μg. specifically up to 280 μg, specifically up to 285 μg, specifically up to 290 μg, specifically up to 295 μg, specifically up to 300 μg, specifically up to 305 μg, specifically up to 310 μg, specifically up to 315 μg, specifically up to 320 μg, specifically up to 325 μg, specifically up to 330 μg, specifically up to 335 μg, specifically up to 340 μg, specifically up to 345 μg, specifically up to 350 μg, specifically up to 400 μg, specifically up to 450 μg, specifically up to 500 μg, specifically up to 540 μg, or specifically up to 650 μg.

During any treatment period, the pegylated interferon-α can be administered at a constant dose, meaning that the same dose is administered each time or only minimally different doses are administered (e.g., dose variation or deviation of less than 10%, specifically less than 5%, specifically less than 1%). Alternatively, different doses can be administered at a regular interval during a treatment period. For example, the interferon can be administered at a particular dose at a regular interval for a certain time, and it can then be administered at a different dose (higher or lower than the first dose) at the same regular interval.

The subject can be a subject who has not been treated with an interferon before or a subject who had previously been administered a dose (e.g., 12.5, 15, 18.75, or 25 μg) of a type I interferon once per week or every two weeks. The subject can be a subject who has been treated previously with a therapy other than an interferon (e.g., HU).

A subject in need thereof can be treated with a pegylated interferon-α using one dosage regimen for a time period and then switched to a different dosage regimen.

More specifically, a 50 to 650 μg dose of a pegylated interferon-α can be administered to a subject in need thereof at a first regular interval for a first treatment period, the first interval being 2 to 4 weeks (e.g., 2, 3, or 4 weeks), and subsequently, a 50 to 650 μg dose of the pegylated type I interferon is administered to the subject at a second regular interval for a second treatment period, the second interval being at least 3 weeks (e.g., 3, 4, 5, 6, 7, or 8 weeks).

Subjects who show a good response to a pegylated interferon-α dosage regimen can be switched to another regimen in which the interferon is administered at a lower dose, higher dose, and/or at a longer interval.

In some embodiments, the total amount of the pegylated interferon-α administered to the subject per a given period (e.g., 1 week, 2 weeks, 4 weeks, 1 month, or 2 months) during the second treatment period is lower or higher (e.g., lower or higher by 20%, 30%, 40%, 45%, 50%, or more) than the total amount administered per the same given period during the first treatment period. For example, the monthly total amount of the interferon administered during the second treatment period can be lower or higher (e.g., by 20%, 30%, 40%, 45%, 50%, or more) than the monthly total amount administered during the first treatment period.

The dose administered during the first treatment period and the dose administered during the second treatment period can be the same but at different intervals. Alternatively, the dose administered during the second treatment period can be lower or higher than the dose administered during the first treatment period.

The first treatment period and the second treatment period can separately be at least 1, 2, 3, 4, 5, 6, 7, 8, 9, 10, 11, 12, 24, 36, 42, 48, 54, 60, 66, 72, 78, 84 or more months. In some embodiments, each treatment period is, individually, at least 1, 2, 3, 4, 4.5, 5, 5.5, 6, 6.5, 7, 7.5, 8, 8.5, 9, 9.5, 10 or more years.

The first treatment period can continue until the subject shows a good response to the treatment. Whether a subject is responding well to the treatment can be determined by a practitioner skilled in the art based on art-accepted criteria (e.g., hematological parameter, allelic burden, symptoms, and adverse events).

In any of the methods or treatment periods described herein, the pegylated interferon-α may be titrated. A subject can be treated with a lower starting dose (e.g., 50 to 350 μg) of a pegylated interferon-α every 2 to 8 weeks. If the subject responds well (e.g., lack of significant drug-related adverse events, significant self-reported discomfort, abnormal hematological responses or other symptoms) after a time, the dose given to the subject may be increased incrementally (e.g., by 50 to 150 μg, 50 μg, 75 μg, 100 μg, 125 μg, or 150 μg, or a combination thereof) every 2 to 12 weeks (e.g., every 2, 3, 4, 5, 6, 7, 8, 9, 10, 11, or 12 weeks, or a combination thereof) until the dose reaches a target dose (e.g., at least 400 μg, 425 μg, 450 μg, 475 μg, 500 μg, 525 μg, 550 μg, or 650 μg). After that, the target dose is maintained during the treatment period. The dose can be increased successively until the desired dose is reached. For example, if the pegylated interferon-α is administered once every 2, 3, 4, 5, 6, 7, or 8 weeks, the dose can be increased every 2, 3, 4, 5, 6, 7 or 8 weeks, respectively. For example, a subject may be given a starting dose of 100 μg. If the subject responds well to the initial dose, the dose may be increased by 100 μg every two weeks until it reaches a target dose of 500 μg. In some embodiments, the target dose is reached in 4 to 48 weeks from the first administration of the pegylated interferon-α (e.g., 4 to 12 weeks, 4 to 16 weeks, 4 to 20 weeks, 4 to 24 weeks, 6 to 12 weeks, 6 to 16 weeks, 6 to 20 weeks, 6 to 24 weeks, 6 to 28 weeks, 6 to 32 weeks, 6 to 36 weeks, 6 to 40 weeks, 8 to 12 weeks, 8 to 16 weeks, 8 to 20 weeks, 8 to 24 weeks, 8 to 28 weeks, 8 to 32 weeks, 8 to 36 weeks, 8 to 40 weeks, 12 to 16 weeks, 12 to 20 weeks, 12 to 24 weeks, 12 to 28 weeks, 12 to 32 weeks, 12 to 36 weeks, 12 to 40 weeks, or 12 to 48 weeks). In some embodiments, the target dose is reached in 4 to 8 weeks from the initial administration of the pegylated interferon-α. During the titration process, any dose, prior to reaching the target dose, may be maintained for a time period (e.g., 4 to 16 weeks) or a number of successive doses (e.g., 2 to 8 successive doses) or reduced depending on the subject's response. In some embodiments, the target dose is reached within 2 to 4 successive doses.

An initial dose or starting dose of the pegylated interferon-α refers to the first dose administered to a subject during a treatment period (i.e., week 0), wherein, prior to the treatment period, the subject is interferon-treatment naïve or has not been administered the same pegylated interferon-α. A subject who is interferon-treatment naïve is a subject who has not been treated with any form of interferon, whether pegylated or non-pegylated (e.g., recombinant interferon, or peginterferon alpha-2b or peginterferon alpha-2a approved to be administered weekly).

Response criteria for assessing treatment can include symptoms and signs of the disease, peripheral blood counts (e.g., platelet counts and white blood cell counts), vascular events, signs of progression of disease, bone marrow histology, and molecular response. Response criteria can be defined based on consensus criteria in the art, e.g., the European LeukemiaNet (ELN) and/or International Working Group (IWG) criteria.

For example, any combination of the following criteria can be used to define a response for essential thrombocythemia or polycythemia vera: resolution of disease-related signs including palpable hepatosplenomegaly; large symptoms improvement; platelet count ≤400×10⁹/L; WBC count ≤10×10⁹/L; hematocrit <45% (with or without phlebotomy in the previous 3 months or 12 weeks); absence of leukoerythroblastosis; absence of signs of progressive disease; absence of any hemorrhagic or thrombotic events; bone marrow histological remission (e.g., disappearance of megakaryocyte hyperplasia and absence of >grade 1 reticulin fibrosis; or presence of age-adjusted normocellularity and disappearance of trilinear hyperplasia, and absence of >grade 1 reticulin fibrosis); and molecular remission or response. A complete response (e.g., a complete hematological response) can be defined to include all or a subset of the criteria. A partial response (e.g., a partial hematological response) can be defined to include a smaller subset of the criteria.

For myelofibrosis (MF), e.g., associated with primary MF, post-polycythemia vera MF, and post-essential thrombocythemia MF, any combination of the following criteria can be used to define a response: age-adjusted normocellularity; <5% blasts; ≤grade 1 MF; hemoglobin ≥100 g/L and <UNL; neutrophil count ≥1×10⁹/L and <UNL; platelet count ≥100×10⁹/L and <UNL; <2% immature myeloid cells; resolution of disease symptoms; spleen and liver not palpable; no evidence of extramedullary hematopoiesis (EMH); hemoglobin ≥85 but <100 g/L and <UNL; platelet count ≥50, but <100×10⁹/L and <UNL; achievement of anemia, spleen or symptoms response without progressive disease or increase in severity of anemia, thrombocytopenia, or neutropenia; transfusion-independent patients: a ≥20 g/L increase in hemoglobin level; transfusion-dependent patients: becoming transfusion-independent; a baseline splenomegaly palpable at 5-10 cm, below the LCM, becomes not palpable; a baseline splenomegaly palpable at >10 cm, below the LCM, decreases by ≥50%; ≥35% spleen volume reduction; ≥50% reduction in the MPN-SAF TSS; cytogenic remission or response; and molecular remission or response.

A molecular response can include a reduction of a mutant allele burden. For example, a molecular response can include a reduction in JAK2617F allele burden.

Allele burden (%) over time can be calculated. Allelic burden represents the percentage of mutant alleles present among all alleles of a particular gene in peripheral blood mononuclear cells. More specifically, the reduction of the allele burden can be at least 20%, 25%, 30%, 35%, 40%, 45%, 50%, 55%, 60%, 65%, 70%, 75%, 80%, 85%, 90%, 95%, or 99% or more, specifically between two time points or within a treatment period. The allele burden can decline to 50% or less, e.g., less than 50%, 45% or less, 40% or less, 37% or less, 35% or less, 30% or less, 25% or less, 20% or less, 15% or less, 10% or less, 7% or less, 5% or less, or 1% or less. A complete molecular response (CMR) is achieved when the allele burden is below the threshold of 1%. In some embodiments, the allele burden may be reduced to less than 0.01%.

Other indications of a good response can include a normal spleen size (measured via ultrasound measured via ultrasound; ≤12 cm for females, ≤13 cm for males), absence or low rate of any thromboembolic events, and a reduction of phlebotomy requirements by at least 50%, e.g., at least 55%, 60%, 65%, 70%, 75%, 80%, 85%. 90%, 95%, or 99%. A patient can be free of phlebotomy.

Complete hematologic response (CHR) can include hematocrit <45% (without phlebotomy in the previous 3 months), platelet count <400×10⁹/L, white blood cell count <10×10⁹ cells/L, with or without normal spleen size, and absence of thromboembolic events.

In some cases, the treated subject exhibits one or both of CHR and CMR during or at the end of a treatment period. The subject may be also free of phlebotomy and/or has a normal spleen size.

For example, any of the above responses (e.g., CHR and CMR) can occur or be detected in a subject by month 3, 6, 12, 18, 24, 30, 36, 42, 48, 52 or 60 or by year 1, 2, 3, 4, 5, or 6 after initiation of the treatment. Any of the responses can be maintained or further improved throughout the treatment period or beyond.

In a group of subjects (e.g., at least 10 subjects, 30 to 500 subjects, or at least 50 subjects) treated with a conjugate of formula I (treatment group), the rate of CHR and molecular response (complete or partial) at the end of a given period can be higher (e.g., 5% to 100% higher) than in a control group of subjects treated with HU or a different pegylated interferon at the end of the same given period. Alternatively or in addition to, the treatment group can have a higher molecular response rate (e.g., 5% to 100% higher) than the control group, or can have a lower median allele burden (e.g., 5% to 60% lower) than the control group. The treated group may also have a longer median duration of maintenance of CHR (with or without normal spleen size), e.g., at least 2 to 36 months longer, or at least 6, 12, 18, 24, 30, 36, 42, 48, 54, or 60 months longer, than the control group. The treated group may have one or more subjects exhibiting one or both of CHR and CMR. The given period for comparison may be 1, 2, 3, 4, 5, 6, 7, 8, or more years.

A subject treated with a conjugate of formula I can exhibit less or similarly frequent adverse events (e.g., 5% to 100%, 10% to 30%, 20% to 40%, 30% to 50%, 40% to 60%, or 50% to 70% less of total adverse events, any adverse events, ≥grade 3 events, or ≥grade 4 events) or similar or lower grade events (e.g., absence of ≥grade 3 events) than a subject treated with a different pegylated interferon or HU.

Adverse events can include hematologic, non-hematologic, or biochemical adverse events. Hematologic adverse events can include anemia, neutropenia, lymphopenia, thrombocytopenia, and pancytopenia. Non-hematologic adverse events can include infections, psychiatric disorders (e.g., depression), asthenia, fatigue, musculoskeletal pain, muscle cramps, abdominal pain, edema, dizziness, rash, headache, nausea, thrombosis, weight gain, weight loss, seizures, hemorrhage, diarrhea, and vomiting. Biochemical events can include elevated aspartate aminotransferase, alanine aminotransferase, and gamma-glutamyltransferase levels. Adverse events are graded based on standards accepted in the field (e.g., National Cancer Institute Common Terminology Criteria for Adverse Events).

The specific example below is to be construed as merely illustrative, and not limitative of the remainder of the disclosure in any way whatsoever. Without further elaboration, it is believed that one skilled in the art can, based on the description herein, utilize the present disclosure to its fullest extent.

### Example: Use of Ropeginterferon Alpha-2b in Polycythemia Vera

### I. Methods

In the PROUD-PV/CONTINUATION-PV study, long-term treatment with ropeginterferon alpha-2b (hereafter ropeg) was compared with standard cytoreductive therapy regarding thromboembolic and other adverse events as well as hematological and molecular parameters over a 5-year period.

Cytoreduction-naïve or HU-pre-treated patients aged ≥18 years diagnosed with PV according to WHO 2008 criteria were eligible. A total of 257 patients were randomly allocated to ropeg or hydroxyurea (HU) at individualized doses for 12 months in the initial study phase (PROUD-PV). The PROUD-PV study was a randomized, open-label, multicenter, controlled, parallel arm, phase III study assessing the efficacy and safety of ropeg versus HU in PV patients.

TABLE 1
Patient characteristics at baseline
All patients enrolled in CONTINUATION-PV

| Characteristics at baseline (in PROUD-PV) | Ropeg (n = 95) | Control (n = 76) |
|---|---|---|
| Caucasian | 100% | 100% |
| WHO2008 PV* | 100% | 100% |
| Female | 48 (50.5%) | 40 (52.6%) |
| Age (median, range) | 58 (30-85) | 59 (32-79) |
| Disease duration (median, range) | 1.8 months | 1.6 months |
| All patients, both HU naïve and pre-treated | (0-146) | (0-92) |
| HU pretreated | 31 (32.3%) | 25 (32.9%) |
| Duration of prior HU treatment (n, median, range) | n = 30 | n = 20 |
| In HU pre-treated patients only | 9.5 months | 8.2 months |
| | (0.9-30.9) | (1.0-36.4) |
| Hematocrit (means, SD) | 48.3 (±5.30) | 42.9 (±23.01) |
| Spleen length (median, range) | 13.5 cm | 12.8 cm |
| | (8.5-25.0) | (7.5-22.0) |
| Clinically significant splenomegaly at baseline** | 7 (7.4%) | 8 (10.5%) |
| Disease-related symptoms at baseline | 15 (15.8%) | 17 (22.4%) |
| Median JAK2V617F burden (range) | 37.3% (2.6-94.9) | 38.1% (2.5-86.6) |

*confirmed by bone marrow biopsy
**investigator assessment

In the ongoing extension phase (CONTINUATION-PV or CONTI-PV), patients in the HU arm were permitted to switch to best available treatment. Efficacy assessments included a longitudinal analysis of complete hematological response and complete molecular response, defined by modified ELN criteria. Discontinued patients were considered non-responders. Data were analyzed for patients reaching 48 months and 60 months of treatment.

Among the patients rolled over from the PROUD-PV to the CONTI-PV study, 95 patients received ropeg and 76 received best available therapy (BAT), e.g., HU. Patients in the ropeg arm self-administered ropeg every 2, 3 or 4 weeks in the home-setting.

At the time of the 4-year analysis, 139 patients remained on study: 74/95 in the ropeg arm and 65/76 in the control arm. Almost all patients in the control arm (>97% at the last available assessment) continued on HU.

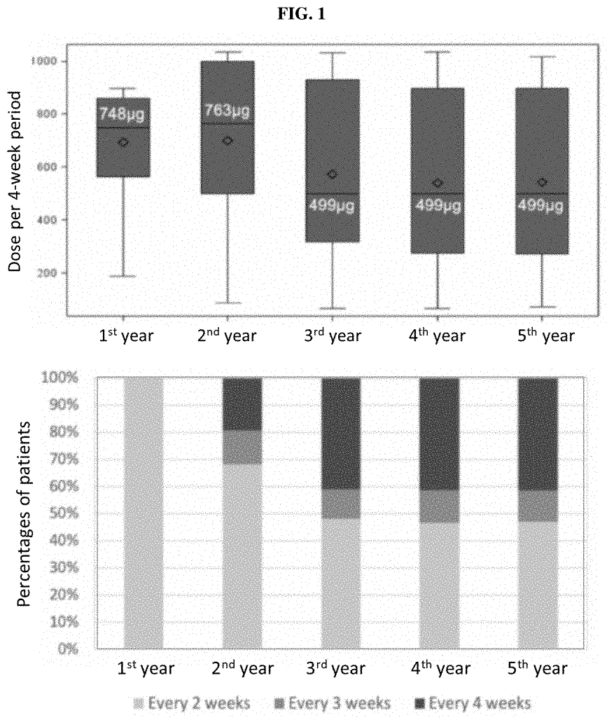

Most patients in the control arm continued to receive HU (88% at month 60). At the time of the 5-year analysis, 70 patients in the ropeg arm and 57 in the control arm remained on study. Discontinuation rates were balanced between the treatment arms (ropeg: 26.3%; control: 25.0%). FIG. 1 shows the doses of ropeginterferon alpha-2b (μg) administered to subjects per 4-week period during the study.

Long-term disease responses were presented as complete hematologic response (CHR) alone or in relation to spleen size. CHR includes hematocrit <45% (without phlebotomy for at least 3 months), platelet count <400×10⁹/L, white blood cell count <10×10⁹ cells/L, with or without normal spleen size (measured via ultrasound; ≤12 cm for females, ≤13 cm for males), and absence of thromboembolic events.

JAK2V617F allelic burden (%) over time was calculated. Allelic burden represents the percentage of mutant JAK2 alleles present among all JAK2 alleles in peripheral blood mononuclear cells. Molecular response was defined as change in the JAK2V617F allele burden after treatment. Criteria for molecular response were categorized into complete (CR), partial (PR) or nonresponse: CR—reduction of allele burden to less than 1%; PR—reduction from baseline value ≥50% in patients with <50% mutant allele burden at baseline or reduction from baseline value ≥25% in patients with ≥50% mutant allele burden at baseline; nonresponse—all other responses. JAK2V617F was determined using real-time PCR with Ipsogen® JAK2 MutaQuant® kit (QIAGEN GmbH).

Efficacy analysis included CHR, spleen normality, and improvement in disease burden such as disease-related signs and symptoms.

### II. Hematologic Responses

The rate of patients in CHR was significantly higher in the ropeg arm than in the control arm in the 4^{th} year (60.6% versus 43.4%; p=0.02), as seen after 2 and 3 years of treatment. In line with this effective control of hematologic parameters by ropeg, a very low rate of major thromboembolic adverse events was observed in the ropeg arm: 0.0%, 0.0% and 1.1% of patients in the 2^{nd}, 3^{rd} and 4^{th} years, respectively. In the control arm, rates of major thromboembolic adverse events in the 2^{nd}, 3^{rd} and 4^{th} year were 0.9%, 1.4% and 0.0%, respectively.

Hematocrit <45% was maintained without the need for phlebotomy in 81.8% of patients in the ropeg arm in the fifth year of treatment, which was significantly higher than the rate of 63.2% observed in the control group (p=0.01). Very few patients experienced a major thromboembolic adverse event (4.2% [1.2%-patient year] of patients in the ropeg arm and 6.6% [1.2%-patient-year] of patients in the control arm during the entire treatment period).

Table 2 shows the rate of CHR over the 5-year study period. Discontinued patients were counted as non-responders. Among the discontinued patients, 68% of those on ropeg showed CHR at their last visits. Only 35% of the discontinued patients in the control group had CHR at the last visits. Table 3 shows the reasons for not meeting definition of CHR at month 60. Table 4 shows the rate of CHR with with last observation carried forward (LOCF).

TABLE 2
Rate of complete hematologic response

| | Responder/ | Responder | Responder/ | Responder | | |
|---|---|---|---|---|---|---|
| Study | N | % | N | % | | RR [95% CI] |
| Month | Ropeg (N = 95) | | Control (N = 76) | | P-value | (Ropeg/Control) |
| 12 | 59/95 | 62.1 | 57/76 | 75.0 | 0.1303 | 0.85 (0.70-1.05) |
| (EOT in PR) | | | | | | |
| 24 | 67/95 | 70.5 | 33/67 | 49.3 | 0.0129 | 1.41 (1.07-1.84) |
| 36 | 67/95 | 70.5 | 38/74 | 51.4 | 0.0104 | 1.39 (1.08-1.78) |
| 48 | 57/94 | 60.6 | 34/76 | 44.7 | 0.0275 | 1.39 (1.04-1.86) |
| 60 | 53/95 | 55.8 | 33/75 | 44.0 | 0.0974 | 1.30 (0.95-1.77) |

TABLE 3
Reasons for not meeting definition of complete hematologic response at month 60

| | Ropeg IFN (N = 95) | Control (N = 76) |
|---|---|---|
| Non-Responder/N (%) | 42/95 (44.2%) | 42/75 (56%) |
| Hematocrit (Hct) | 12 (28.6%) | 9 (21.4%) |
| Platelets | 2 (4.8%) | 4 (9.5%) |
| Leukocytes | — | 3 (7.1%) |
| Hct and platelets | 3 (7.1%) | 3 (7.1%) |
| Platelets and leukocytes | — | 2 (4.8%) |
| Hct and leukocytes | — | 1 (2.4%) |
| Hct, platelets and leukocytes | — | 3 (7.1%) |
| Premature discontinuation N (%) | 25 (59.5%) | 17* (40.5%) |

*N = 17 as of Month 60;
N = 19 as of database lock

TABLE 4
Complete hematologic response with last observation carried forward (LOCF)

| | Ropeg (N = 95) | | Control (N = 76) | | | |
|---|---|---|---|---|---|---|
| Study | Responder | Responder | Responder | Responder | | RR [95% CI] |
| Month | N | % | N | % | P-value | (Ropeg/Control) |
| 12 | 59 | 62.1% | 57 | 75.0% | 0.1 | 0.85 (0.70-1.04) |
| (EOT in PR) | | | | | | |
| 24 | 70 | 73.7% | 43 | 56.6% | 0.04 | 1.27 (1.02-1.60) |
| (LOCF) | | | | | | |
| 36 | 73 | 76.8% | 41 | 54.0% | 0.003 | 1.43 (1.13-1.81) |
| (LOCF) | | | | | | |
| 48 | 68 | 71.6% | 38 | 50.0% | 0.004 | 1.46 (1.13-1.89) |
| (LOCF) | | | | | | |
| 60 | 69 | 72.6% | 40 | 52.6% | 0.004 | 1.43 (1.12-1.81) |
| (LOCF) | | | | | | |

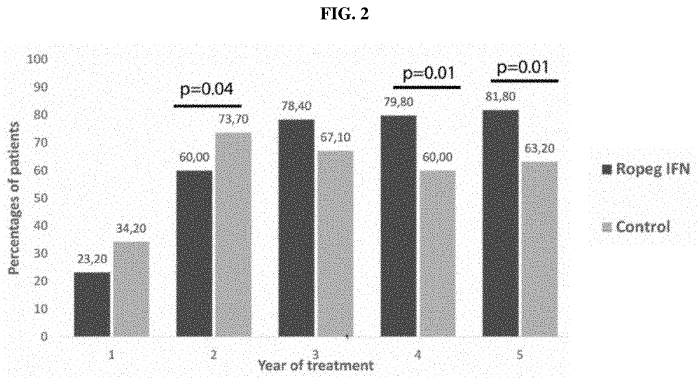

As shown in FIG. 2, at the 4-year and 5-year treatment, a significant greater percentage of patients treated with ropeg was phlebotomy-free as compared to patients in the control group.

In the ropeg arm, the median was not reached for CHR, because fewer than 50% of patients in the ropeg arm who achieved a response lost their response within the 52-month observation period (95% CI, 38.7 to N.A.). In the control arm, the median duration of maintenance of CHR was 17.97 months (95% CI, 11.93 to 21.15). Longer maintenance periods of longest CHR with no increase in spleen size and longest CHR with normal spleen size were also observed in the ropeg group compared with the control group, although the median was reached in the ropeg group for these response parameters

### II. Molecular Responses

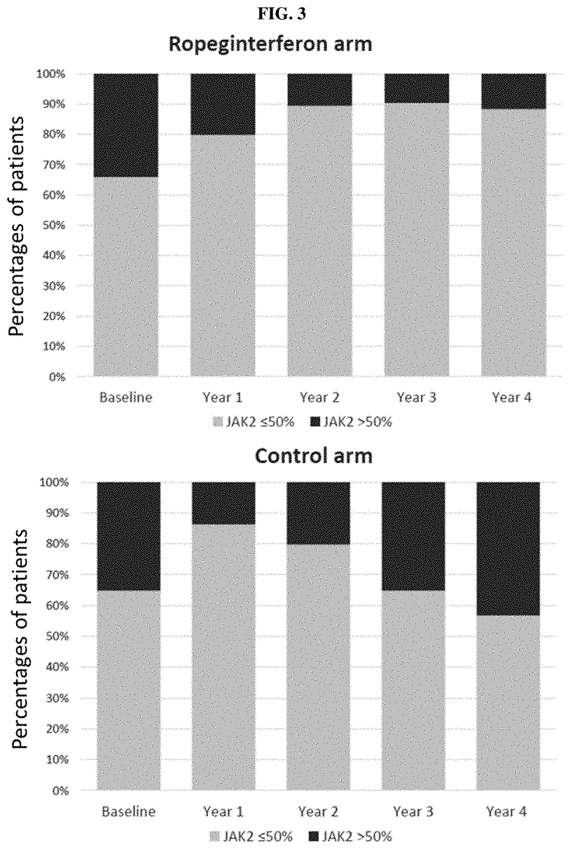

The median JAK2V617F allele burden declined from 37.3% at baseline to 9.8% over 4 years in the ropeg arm, whereas in the control group, the median allele burden increased from 38.1% to 43.1% in the same period (p<0.0001). The rate of molecular response (partial or complete) at 48 months was significantly higher among ropeg-treated patients than in the control arm (67.0% versus 25.7%; RR: 2.5 [95% CI: 1.7 to 3.7; p<0.0001]). No patients achieved CMR in the control arm. In the ropeg arm, 13 patients had a JAK2V617F allele burden below the threshold of 1% at month 48, 11 of whom also had a CHR at this time point. See Table 5. An additional 34 patients in the ropeg arm achieved an allele burden <10% at 48 months. Further, as shown in FIG. 3, the rate of patients with allele burden >50% declined steadily in the ropeg arm. In the control arm, the rate of patients with allele burden >50% rebounded after the first year. Table 6 shows the characteristics of complete molecular responders. Among the 13 responders, the baseline JAK2V617F allele burden ranged from 10% to 62.7%. Among the non-CMR patients, the baseline JAK2V617F allele burden ranged from 2.6% to 94.9%.

TABLE 5
Molecular response at 48 months

| | Ropeg | Control |
|---|---|---|
| Result at 48 months | N/Nmiss = 94/1 | N/Nmiss = 74/2 |
| JAK2V617F ≥1 to <10% | 34 (36.2%) | 8 (10.8%) |
| JAK2V617F ≥1 to <10% and CHR | 24 (25.5%) | 5 (6.8%) |
| JAK2V617F <1% | 13 (13.8%) | — |
| JAK2V617F <1% and CHR | 11 (11.7%) | — |

TABLE 6
Baseline characteristics of complete molecular responders at 48 months

| | | Ropeginterferon arm | |
|---|---|---|---|
| Baseline | | CMR | No CMR |
| characteristic | Statistics | N = 13 | N = 82* |
| Age [years] | Mean | 54.3 | 57.9 |
| | Range | 45.0-65.0 | 30.0-85.0 |
| Gender | Female (%) | 61.5 | 48.8 |
| Time since PV | Median | 2.0 | 1.7 |
| diagnosis [month] | Range | 0.0-11.0 | 0.1-145.5 |
| Prior HU treatment | % | 38.4 | 30.4 |
| JAK2V617F (%) | Median | 36.8 | 37.6 |
| | Range | 10.0-62.7 | 2.6-94.9 |
| Dose of ropeginterferon | Mean | 375 | 349 |
| at 48 months | Median | 375 | 400 |
| | Range | 250-500 | 50-500 |

The median JAK2V617F allele burden declined from 37.3% at baseline to 7.3% over 5 years of treatment in the ropeg arm, whereas in the control arm the median allele burden increased from 38.1% to 42.6% in the same period (p<0.0001). Also see Table 7. The rate of molecular response at 5 years was also significantly higher among ropeg-treated patients than in the control arm (69.1% versus 21.6%; RR: 3.2 [95% CI: 2.1 to 4.9; p<0.0001]). See Table 8. Table 9 shows allele burden at month 60 using different thresholds. In the ropeg group, 10 patients had allele burden below 1% and 5 patients had allele burden below 0.01%, 14 of whom also had CHR.

TABLE 7
JAK2V617F allele burden with last observation carried forward (LOCF)

| Study | Ropeg (N = 95) | | Control (N = 76) | | | RR [95% CI] |
|---|---|---|---|---|---|---|
| Month | Mean | Median | Mean | Median | P-value | (Ropeg/Control) |
| Baseline | 42.8 | 37.3 | 42.9 | 38.1 | — | — |
| 12 | 30.2 | 24.4 | 24.4 | 18.2 | 0.0244 | 6.646 (0.86 to 12.43) |
| 24 | 20.9 | 14.3 | 32.4 | 25.1 | 0.0003 | −10.745 (−16.50 to −4.98) |
| 36 | 19.7 | 11.3 | 39.3 | 40.5 | <0.0001 | −18.722 (−24.49 to −12.96) |
| 48 | 19.3 | 9.2 | 44.8 | 44.2 | <0.0001 | −24.582 (−30.35 to −18.82) |
| 60 | 18.9 | 8.5 | 44.0 | 44.4 | <0.0001 | −23.959 (−29.72 to −18.20) |

TABLE 8
Rate of molecular response with last observation carried forward (LOCF)

| | Ropeg (N = 95) | | Control (N = 76) | | | |
|---|---|---|---|---|---|---|
| Study | Responder/ | Responder | Responder/ | Responder | | RR [95% CI] |
| Month | N | % | N | % | P-value | (Ropeg/Control) |
| 12 | 41/94 | 43.6 | 36/73 | 49.3 | 0.3744 | 0.87 [0.63-1.19] |
| (EOT in PR) | | | | | | |
| 24 | 64/94 | 68.1 | 24/74 | 32.4 | 0.0001 | 2.00 [1.41-2.84] |
| (LOCF) | | | | | | |
| 36 | 62/94 | 66.0 | 20/74 | 27.0 | <0.0001 | 2.31 [1.56-3.43] |
| (LOCF) | | | | | | |
| 48 | 63/94 | 67.0 | 19/74 | 25.7 | <0.0001 | 2.50 [1.67-3.73] |
| (LOCF) | | | | | | |
| 60 | 65/94 | 69.1 | 16/74 | 21.6 | <0.0001 | 3.04 [1.96-4.71] |
| (LOCF) | | | | | | |

TABLE 9
Molecular response at 60 months

| | Ropeg IFN | Control | |
|---|---|---|---|
| Allele burden category at 60 months* | N = 70 | N = 57 | |
| JAK2V617F <0.01% (undetectable**): | N = 5 | N = 0 | |
| Range of baseline JAK2 values | 14-56% | NA | |
| JAK2V617F undetectable and CHR | 4/5 | NA | |
| JAK2V617F ≥0.01% to <1%: | N = 10 | N = 1 | |
| Range of baseline JAK2 values | 10-75% | 47% | |
| JAK2V617F ≥0.01% to <1% and CHR | 9/10 | 1/1 | |
| JAK2V617F≥1% to <10%: | N = 22 | N = 6 | |
| Range of baseline JAK2 values | 19-84% | 12-41% | |
| JAK2V617F ≥1 to <10% and CHR | 19/22 | 3/6 | |
| Total patients with JAK2V617F <10% | N = 37 | N = 7 | p<0.0001 |
| | 53% | 12% | |

*Only patients with a baseline value ≥10% were included in the analysis
**Below limit of detection using the Ipsongen ® JAK2 MutaQuant ® kit

The sustained molecular response observed in ropeg-treated patients was accompanied by a low risk of disease progression. Only 1 case of progression to myelofibrosis (0.20%-patient year) was reported during the entire study period and no leukemic transformation occurred. In contrast, 2 cases of progression to myelofibrosis and 2 cases of transformation to acute leukemia (1.0%-patient year in total) were reported in the control arm.

A further analysis of combined hematologic and molecular parameters was performed, these being known to influence the risk of thrombosis and of progression in PV. At the 5 year visit, 58.5% of patients receiving ropeg had well-controlled hematocrit (<45%) without requiring phlebotomy, as well as achieving a molecular response, compared to 17.3% on standard treatment (RR: 3.52 [2.13 to 5.81]; p<0.0001). See Table 10.

TABLE 10
Combined analysis of hematologic and molecular parameters

| | Responder/ | Responder | Responder/ | Responder | | |
|---|---|---|---|---|---|---|
| Study | N | % | N | % | RR [95% CI] | |
| Month | Ropeg (N = 95) | | Control (N = 76) | | (Ropeg/Control) | P-value |
| 12 | 31/94 | 33.0% | 33/73 | 45.2% | 0.73 (0.50-1.06) | 0.0943 |
| (EOT in PR) | | | | | | |
| 24 | 52/94 | 55.3% | 18/72 | 24.3% | 2.26 (1.46-3.50) | 0.0003 |
| (LOCF) | | | | | | |
| 36 | 54/94 | 57.5% | 19/74 | 25.7% | 2.17 (1.43-3.29) | 0.0003 |
| (LOCF) | | | | | | |
| 48 | 54/94 | 57.5% | 15/74 | 20.3% | 2.79 (1.74-4.46) | <0.0001 |
| (LOCF) | | | | | | |
| 60 | 55/94 | 58.5% | 13/75 | 17.3% | 3.26 (1.97-5.42) | <0.0001 |
| (LOCF) | | | | | | |

### III. Safety

In terms of safety, no new signals were detected in the 4^{th} year. See Table 11. Rates of patients with treatment-related adverse events remained similar in the ropeg and control arms in the 4^{th} year (ropeg: 28.7% of patients; control: 22.9%). Over the entire treatment period, the rates of grade ≥3 drug-related adverse events were similar in both arms. Most common treatment-related adverse events included thrombocytopenia, leukopenia, anemia, and elevated alanine aminotransferase and gamma-glutamyltransferase levels. Disease or treatment-related secondary malignancies reported in the entire study period comprised 2 cases of acute leukemia, 2 cases of basal cell carcinoma and 1 case of malignant melanoma, all in the control group. One case of disease-related transformation to myelofibrosis occurred in each treatment arm. Class reactions associated with interferon were also assessed by investigators. See Table 12.

TABLE 11
Summary of safety profile (entire 4-year study period)

| | Patients with adverse event | |
|---|---|---|
| | Ropeginterferon | Control |
| | N = 127 | N = 127 |
| Adverse events (AEs) | 89.8% | 91.3% |
| Treatment-related AEs | 75.6% | 78.7% |
| Treatment-related AEs ≥ grade 3 | 15.0% | 16.5% |
| Serious adverse events (SAEs) | 20.5% | 21.3% |
| Treatment related SAEs | 2.4% | 5.9% |

TABLE 12
Treatment-related adverse events of special interest to interferon therapy

| | Number of patients with treatment-related event (AE) | |
|---|---|---|
| System Organ Class | in Ropeginterferon arm (N = 127) | |
| (SOC) category | Total for SOC | Individual terms |
| Endocrine disorders | 5 (3.9%) | Autoimmune thyroiditis (n = 1) |
| | | Hypothyroidism (n = 3) |
| | | Hyperthyroidism (n = 1) |
| Psychiatric disorders | 1 (0.8%) | Depression (n = 1), Anxiety (n = 1) |
| | | Mood altered (n = 1), Nervousness (n = 1) |
| Musculoskeletal/ | 2 (1.6%) | Rheumatoid arthritis (n = 1) |
| connective tissue disorders | | Sjogren’s syndrome (n = 1) |
| Skin/subcutaneous | 2 (1.6%) | Dermatitis acneiform (n = 1) |
| tissue disorders | | Psoriasis (n = 1) |
| Immune system disorders | 1 (0.8%) | Sarcoidosis (n = 1) |

No new signals were detected in the fifth year. See Table 13. Treatment-related adverse events were reported in 25.6% and 24.2% of patients in the ropeg and control arms, respectively, and one patient in each arm withdrew due to drug-related toxicity. Three patients (3.8%) in the ropeg arm reported grade ≥3 treatment-related adverse events in the fifth year. Over the entire treatment period, the rate of grade ≥3 drug-related adverse events was the same in each study arm (16.5%). Over the entire 5 year treatment period, 6 major thromboembolic events occurred in the ropeg arm and 5 events occurred in the control arm. Also, over the entire treatment period, one case of transformation to myelofibrosis occurred in the ropeg arm, and 2 cases of myelofibrosis and two cases of acute leukemia occurred in the control arm. Adverse events of special interest to interferon therapy were assessed by investigators. Compared to the 4^{th} year data, there was only one additional case of hypothyroidism.

TABLE 13
Summary of safety profile

| | Entire treatment period | | Fifth year of treatment | |
|---|---|---|---|---|
| | Ropeg IFN | Control | Ropeg IFN | Control |
| | (N = 127) | (N = 127) | (N = 78) | (N = 66) |
| Adverse | 116 | 117 | 45 | 45 |
| events (AEs) | 91.3% | 92.1% | 57.7% | 68.2% |
| Serious adverse | 30 | 32 | 8 | 5 |
| events (SAEs) | 23.6% | 25.2% | 10.3% | 7.6% |
| Treatment- | 4 | 5 | 1 | 0 |
| related SAEs | 3.1% | 3.9% | 1.3% | 0 |
| Adverse drug | 100 | 100 | 20 | 16 |
| reactions (ADRs) | 78.7% | 78.7% | 25.6% | 24.2% |
| Grade 3, 4 or 5 ADRs | 21 | 21 | 3 | 0 |
| | 16.5% | 16.5% | 3.8% | 0 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A method of treating a myeloid neoplasm in a subject, the method comprising administering to a subject in need thereof a 50 to 500 μg dose of a pegylated interferon-α once every 2 to 8 weeks, the pegylated interferon-α being a conjugate of formula I: 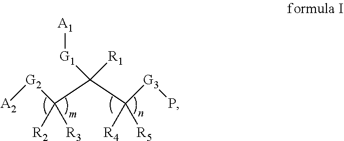
in which
each of R₁, R₂, R₃, R₄, and R₅, independently, is H, C₁₋₅ alkyl, C₂₋₅ alkenyl, C₂₋₅ alkynyl, aryl, heteroaryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10;
wherein the subject has a complete molecular response, the complete molecular response including a JAK2617F allele burden below 1%.

2. The method of claim 1, wherein the JAK2617F allele burden is below 0.01%.

3. The method of claim 1, wherein the subject has the complete molecular response by 24 to 60 months of being treated with the pegylated interferon-α.

4. The method of claim 3, wherein the subject has the complete molecular response by 24 to 48 months of being treated with the pegylated interferon-α.

5. The method of claim 1, wherein the subject has a complete hematological response by 24 to 60 months of being treated with the pegylated interferon-α.

6. The method of claim 5, wherein the subject has the complete molecular response and the complete hematological response simultaneously.

7. The method of claim 5, wherein the complete hematological response includes hematocrit <45% without phlebotomy for at least 3 months, platelet count <400×10⁹/L, and white blood cell count <10×10⁹ cells/L.

8. The method of claim 1, wherein the subject has a JAK2617F allele burden of at least 10% to 60% prior to the administration of the pegylated interferon-α.

9. The method of claim 1, wherein the subject is continuously treated with the pegylated interferon-α after exhibiting the complete molecular response.

10. The method of claim 9, wherein, after the complete molecular response is achieved or detected, the complete molecular response is maintained for at least one year or further improved.

11. The method of claim 1, wherein the subject is treated with the pegylated interferon-α for at least 4 years.

12. The method of claim 11, wherein the subject is treated with the pegylated interferon-α for at least 5 to 10 years.

13. The method of claim 1, wherein the subject is administered the pegylated interferon-α once every 2 to 4 weeks.

14. The method of claim 13, wherein the subject is administered a 250 to 500 μg dose of the pegylated interferon-α once every 2 to 4 weeks.

15. The method of claim 1, wherein the subject is administered a starting dose of 50 to 350 μg of the pegylated interferon-α once every 2 to 8 weeks, and wherein the starting dose is increased incrementally until a target dose of 500 μg is reached.

16. The method of claim 15, wherein the target dose is reached by 4 to 48 weeks from administration of the starting dose.

17. The method of claim 16, wherein the target dose is reached by 4 to 8 weeks from administration of the starting dose.

18. The method of claim 1, wherein the subject is initially administered the pegylated interferon-α once every 2 weeks for a first treatment period and switched to administration of the pegylated interferon-α once every 3 to 8 weeks for a second treatment period.

19. The method of claim 18, wherein the subject has the complete molecular response before or after the switch.

20. The method of claim 1, wherein the myeloid neoplasm is polycythemia vera, essential thrombocythemia or myelofibrosis.

21. The method of claim 20, wherein the myeloid neoplasm is polycythemia vera.

22. The method of claim 1, wherein the conjugate has one or more properties including:
(i) a median Tmax in the range of 3 to 6 days following administration of multiple 50 to 540 μg doses of the conjugate once every two weeks to subjects;
(ii) a mean T_{1/2} in the range of 6 to 10 days following administration of multiple 50 to 540 μg doses of the conjugate once every two weeks to subjects; and
(iii) an individual maximum tolerated dose of at least 500 μg once every 2 to 4 weeks in subjects.

23. The method of claim 22, wherein the conjugate has one or more features including: G3 is a bond and P is an interferon-α moiety in which the amino group at the N-terminus is attached to G3; A₁ and A₂ are polyalkylene oxide moieties each having a molecular weight of 10-30 kD; each of G₁ and G₂ is 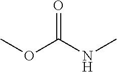
in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues.

24. The method of claim 23, wherein the interferon-α moiety is a human interferon-α-2b having an extra proline residue at the N-terminus and is 166 amino acids in length.

25. The method of claim 23, wherein the conjugate is 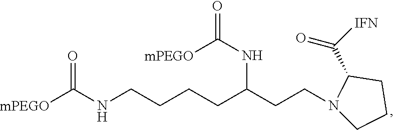
in which mPEG has a molecular weight of 20 kD and IFN is an interferon-α-2b.
